(19) United States
(12) Patent Application Publication
(10) Pub. No.: US 2005/0191259 A1
(43) Pub. Date: Sep. 1, 2005

(54) Composition for coating keratin fibers, comprising at least one tacky microcrystalline wax and fibers

(76) Inventor: Sue Feng, Edison, NJ (US)

Correspondence Address: Thomas L. Irving;FINNEGAN, HENDERSON, FARABOW, GARRETT & DUNNER, L.L.P., 901 New York Avenue, N.W., Washington, DC, 20001-4413, US

(21) Appl. No.: 11/056,261

(22) Filed: Feb. 14, 2005

Related U.S. Application Data
(60) Provisional application No. 60/543,997, filed on Feb. 13, 2004.

(51) Int. Cl. A61K 7/32; A61K 7/06

(52) U.S. Cl. 424/70.1

(57) ABSTRACT

The present disclosure relates to a composition for coating keratin fibers, comprising, in a cosmetically acceptable medium, a microcrystalline wax with a tack of greater than or equal to 0.7 N.s and, for instance, a hardness of less than or equal to 3.5 MPa, and fibers. The disclosure also relates to a process for making up or caring for keratin fibers comprising applying the compositions to keratin fibers, as well as a packaging assembly for the compositions.

This application claims benefit of U.S. Provisional Application No. 60/543,997, filed Feb. 13, 2004, the content of which is incorporated herein by reference.

The present disclosure relates to a cosmetic composition for coating keratin fibers, comprising at least one “tacky” microcrystalline wax and fibers. The present disclosure also concerns a cosmetic process for making up or treating keratin fibers such as the eyelashes, the eyebrows or the hair. The present disclosure additionally relates to a care or makeup composition for the eyes, such as an eyeliner or an eyeshadow.

The composition according to the present disclosure may be a makeup composition for keratin fibers, a makeup base for keratin fibers, or base coat, a composition to be applied onto a makeup, also known as a top coat, or a composition for treating keratin fibers, also known as a base coat. For example, in one embodiment, the composition according to the present disclosure is a mascara.

As used herein, the term “mascara” is understood to mean a composition intended to be applied to the eyelashes: it can be a makeup composition for the eyelashes, a makeup base for the eyelashes, a composition to be applied onto a mascara, also known as a topcoat, or a cosmetic treatment composition for the eyelashes. For example, mascara is can be intended for the eyelashes of human beings, and also for false eyelashes.

Mascaras are commonly prepared according to one of two types of formulation: water-based mascaras, known as cream mascaras, in the form of an emulsion of waxes in water, and anhydrous mascaras or mascaras with a low water content, known as waterproof mascaras, in the form of dispersions of waxes in organic solvents. It is known practice to use various waxes to formulate mascaras, for instance those described in International Patent No. WO-A-91/12793, for example beeswax, candelilla wax, carnauba International Patent No. WO-A-91/12793, for example beeswax, candelilla wax, carnauba wax or polyethylene wax.

However, when mascaras contain certain waxes in high content (for instance, in an amount of greater than 10%), for instance carnauba wax, rice bran wax or polyethylene wax, the makeup of the eyelashes obtained can look grainy, and may thus give a non-smooth and non-uniform makeup result. Such defects can render the makeup result unattractive.

Moreover, to obtain a mascara with good charging properties, i.e. to obtain heavy makeup of the eyelashes, it is possible to incorporate into the mascara at least one wax in a total amount of greater than 25% by weight, relative to the total weight of the mascara. However, when using conventional waxes such as beeswax, candelilla wax or carnauba wax at these high amounts, the mascara composition can acquire a very thick consistency, or can even become too compact, and may not be applied easily to the eyelashes with the mascara brush applicators commonly used. The excessively thick mascara may be deposited on the eyelashes in the form of lumps and the makeup result thus obtained may not have the desired smooth appearance. The makeup result may not be uniform and can look unattractive.

In addition, certain waxes such as orange wax or lanolin wax, used in an amount of greater than 25% by weight, can produce compositions that are not sufficiently stable; for example, after storage for two weeks at room temperature (25° C.): the composition can set to a solid (substantial increase in viscosity) or can undergo a phase separation that may be seen with the naked eye. The composition may then be unsuitable for application to the eyelashes.

Moreover, another property desired in mascara is lengthening of the eyelashes. To obtain such an effect, it is known practice to use fibers as additives. However, the cosmetic compositions of the prior art comprising fibers can have a certain number of drawbacks, such as non-uniform and poorly charging (poorly volumizing) makeup. For instance, mascara compositions comprising fibers of the prior art frequently do not allow an optimum lengthening effect to be obtained on the eyelashes after application of the composition, either on account of poor orientation and a random distribution of the fibers on the eyelashes (which may not lie in the continuation of the eyelashes), or on account of poor attachment to the eyelashes, since the fibers slide on the eyelashes and, in the end, only a small amount of fibers in the product is deposited on the support. The effect obtained by the mascara compositions comprising fibers of the prior art is thus often aesthetically unacceptable, such as in the case of dense and/or long and/or curled eyelashes, for which an unattractive “Christmas tree” appearance of the eyelashes can be obtained, with a non-smooth appearance of the eyelashes.

Another major drawback associated with the use of fibers in the compositions of the prior art is the great difficulty there can be in obtaining a volumizing (charging) effect, since the attachment of the product to the eyelashes is countered by the sliding of the fibers on the surface of the eyelashes.

Accordingly, one aspect of the present disclosure relates to a composition for coating keratin fibers, making it possible to obtain a smooth, uniform makeup result on the keratin fibers, with good, charging distribution of the fibers along the eyelashes. The inventors have discovered, surprisingly and unexpectedly, that this composition may be obtained by using at least one microcrystalline wax that has tacky properties (high tack) and fibers. This wax can produce a composition for coating keratin fibers, such as a mascara, that can apply easily to the eyelashes, can show good attachment to the eyelashes, and can lead to the formation of a smooth, uniform makeup result with a lengthening and charging (volumizing) effect on the eyelashes.

For example, in one embodiment, the present disclosure relates to a composition for coating keratin fibers, comprising, in a cosmetically acceptable medium, fibers and at least one microcrystalline wax with a tack of greater than or equal to 0.7 N.s and, for instance, a hardness of less than or equal to 3.5 MPa. As used herein, the term “microcrystalline wax” is understood to mean a wax derived from petroleum and having crystals that are finer than the crystals of paraffin wax.

Another aspect of the present disclosure is also a non-therapeutic cosmetic makeup or care process for keratin fibers, such as the eyelashes, comprising the application to the keratin fibers of a composition as defined above.

Still another aspect of the present disclosure is the use of a composition as defined above to obtain a uniform and/or smooth makeup result on keratin fibers, such as the eyelashes, and a good lengthening effect on made-up keratin fibers and/or a volumizing effect.

Yet another aspect of the present disclosure is the use of the combination of at least one microcrystalline wax with a tack of greater than or equal to 0.7 N.s and, for instance, a hardness of less than or equal to 3.5 MPa, combined with fibers in a composition for making up keratin fibers, to obtain a uniform and/or smooth makeup result on the keratin fibers, and a lengthening and/or volumizing effect on made-up keratin fibers.

As used herein, the term “cosmetically acceptable medium,” is understood to mean a cosmetic medium that is compatible with the eyelashes or the skin.

### BRIEF DESCRIPTION OF THE FIGURE

FIG. 1 is a representation of a packaging and applicator assembly for coating keratin fibers as disclosed herein.

### MICROCRYSTALLINE WAX

As used herein, the term “wax,” is understood to mean a lipophilic fatty compound that is solid at room temperature (25° C.) and atmospheric pressure (760 mmHg, i.e. 10⁵ Pa), with a reversible solid/liquid change of state, having a melting point of greater than 30° C., for example, greater than 55° C., which may be up to 200° C., for instance up to 120° C.

By bringing the wax to its melting point, it is possible to make it miscible with oils and to form a microscopically homogeneous mixture, but upon cooling the mixture to room temperature, recrystallization of the wax in the oils of the mixture is obtained.

For the purposes of the present disclosure, the melting point is the temperature of the most endothermic peak observed by thermal analysis (DSC) as described in ISO standard 11357-3; 1999. The melting point of the wax may be measured using a differential scanning calorimeter (DSC), for example the calorimeter sold under the name “MDSC 2920” by the company TA Instruments. The measurement protocol is as follows: a sample of 5 mg of wax placed in a crucible is subjected to a first temperature increase ranging from −20° C. to 100° C., at a heating rate of 10° C./minute, it is then cooled from 100° C. to −20° C. at a cooling rate of 10° C./minute and is finally subjected to a second temperature increase ranging from −20° C. to 100° C. at a heating rate of 5° C./minute. During the second temperature increase, the variation of the difference in power absorbed by the empty crucible and by the crucible containing the sample of product is measured as a function of the temperature. The melting point of the compound is the temperature value corresponding to the top of the peak of the curve representing the variation in the difference in absorbed power as a function of the temperature.

The at least one tacky microcrystalline wax present in the composition as disclosed herein has a tack of greater than or equal to 0.7 N.s, for instance, ranging from 0.7 N.s to 30 N.s, such as greater than or equal to 0.8 N.s, for example, ranging from 0.8 N.s to 10 N.s, such as ranging from 0.8 N.s to 5 N.s.

The at least one microcrystalline wax of the composition as disclosed herein can also have, for example, a hardness of less than or equal to 3.5 MPa, such as ranging from 0.01 to 3.5 MPa, for instance, ranging from 0.05 MPa to 3 MPa, from 0.1 MPa to 2 MPa, and from 0.2 to 1 MPa.

The tack of the at least one microcrystalline wax is measured at 20° C. using the texturometer sold under the name TA-XT2i by the company Rheo, equipped with an acrylic polymer spindle in the form of a cone forming an angle of 45°, by measuring the change in force (compression force or stretching force) (F) as a function of time, during the following operation: The spindle is displaced at a speed of 0.5 mm/s and then penetrates into the wax to a penetration depth of 2 mm. When the spindle has penetrated the wax to a depth of 2 mm, the spindle is held stationary for 1 second (corresponding to the relaxation time) and is then withdrawn at a speed of 0.5 mm/s. During the relaxation time, the force (compression force) decreases greatly until it becomes zero, and then, during the withdrawal of the spindle, the force (stretching force) becomes negative before rising again to the value 0. The tack corresponds to the integral of the curve of the force as a function of time for the portion of the curve corresponding to the negative force values (stretching force). The tack value is expressed in N.s.

To perform the tack measurement of the wax, the wax is melted at a temperature equal to the melting point of the wax +10° C. The molten wax is poured into a container 25 mm in diameter and 20 mm deep. The wax is recrystallized at room temperature (25° C.) for 24 hours such that the surface of the wax is flat and smooth, and the wax is then kept for at least 1 hour at 20° C. before performing the tack measurement.

The hardness is determined by measuring the compression force, which is measured at 20° C. using the texturometer sold under the name TA-XT2i by the company Rheo, equipped with a cylindrical stainless-steel spindle 2 mm in diameter, by measuring the change in force (compression force or stretching force) (F) as a function of time, during the following operation: The spindle is displaced at a speed of 0.1 mm/s and then penetrates into the wax to a penetration depth of 0.3 mm. When the spindle has penetrated the wax to a depth of 0.3 mm, the spindle is held stationary for 1 second (corresponding to the relaxation time) and is then withdrawn at a speed of 0.1 mm/s. During the relaxation time, the force (compression force) decreases greatly until it becomes zero, and then, during the withdrawal of the spindle, the force (stretching force) becomes negative before rising again to the value 0. The hardness corresponds to the maximum compression force measured between the surface of the spindle and the wax at the moment that they come into contact. The value of this force is expressed in MPa.

To perform the hardness measurement, the wax is melted at a temperature equal to the melting point of the wax +20° C. The molten wax is poured into a container 30 mm in diameter and 20 mm deep. The wax is recrystallized at room temperature (25° C.) for 24 hours and is then stored for at least 1 hour at 20° C. before performing the hardness measurement.

A non-limiting example of a tacky microcrystalline wax that may be used is the microcrystalline wax sold under the reference SP18 by the company Strahl and Pitsch, which has a hardness of 0.46 MPa and a tack value of about 1 N.s.

The at least one microcrystalline wax can be present in the composition as disclosed herein in an amount ranging from 0.5% to 60%, for instance, ranging from 2% to 40%, such as ranging from 5% to 20% by weight, relative to the total weight of the composition.

Fibers

As used herein, the term “fiber” is understood to mean an object of length L and diameter D such that L is greater than D, for instance, L may be very much greater than D, wherein D is the diameter of the circle in which the cross section of the fiber is inscribed. For example, the ratio of L to D (or shape factor) can range from 3.5 to 2,500, for instance, from 5 to 500, such as from 5 to 150.

The fibers that can be used in the composition of the present disclosure may be mineral or organic fibers of synthetic or natural origin. They may be short or long, individual or organized, for example braided, and hollow or solid. They may have any shape, and may have, for example, a circular or polygonal (square, hexagonal or octagonal) cross section, depending on the intended specific application. In one embodiment of the present disclosure, the fibers' ends are blunt and/or polished to prevent injury.

For example, the fibers can have a length ranging from 1 μm to 10 mm, for instance from 0.1 mm to 5 mm, such as from 0.3 mm to 3 mm. Their cross section may be within a circle of diameter ranging from 2 nm to 500 μm, for instance ranging from 100 nm to 100 μm, such as from 1 μm to 50 μm. The weight or yarn count of the fibers is often given in denier or decitex, and represents the weight in grams per 9 km of yarn. For example, the fibers according to the present disclosure may have a yarn count ranging from 0.01 denier to 10 denier, for instance from 0.1 denier to 2 denier, such as from 0.3 denier to 0.7 denier.

The fibers that can be used include those used in the manufacture of textiles, such as silk fiber, cotton fiber, wool fiber, flax fiber, cellulose fiber extracted for instance, from wood, from legumes or from algae, rayon fiber, polyamide (Nylon®) fiber, viscose fiber, acetate fiber, such as rayon acetate fiber, acrylic polymer fiber, for instance: polymethyl methacrylate fiber or poly(2-hydroxyethyl methacrylate) fiber, polyolefin fiber such as polyethylene or polypropylene fiber, glass fiber, silica fiber, carbon fiber, for instance in graphite form, polytetrafluoroethylene (such as Teflon®) fiber, insoluble collagen fiber, polyester fiber, polyvinyl chloride fiber or polyvinylidene chloride fiber, polyvinyl alcohol fiber, polyacrylonitrile fiber, chitosan fiber, polyurethane fiber, polyethylene phthalate fiber, and fibers formed from a mixture of polymers such as those mentioned above, for instance polyamide/polyester fibers.

In one embodiment of the present disclosure, the fibers are polyamide (Nylon®) fibers.

The fibers used in surgery can also be used, for instance the resorbable synthetic fibers prepared from glycolic acid and caprolactone (Monocryl from Johnson & Johnson); resorbable synthetic fibers of the type which is a copolymer of lactic acid and of glycolic acid (Vicryl from Johnson & Johnson); polyterephthalic ester fibers (Ethibond from Johnson & Johnson) and stainless steel threads (Acier from Johnson & Johnson).

Moreover, the fibers may or may not be surface-treated, and may or may not be coated with a protective coat. Among coated fibers that may be used as disclosed herein, non-limiting mention may be made of polyamide fibers coated with copper sulphide to give an anti-static effect (for example R-STAT from Rhodia) or another polymer enabling a particular organization of the fibers (specific surface treatment). Non-limiting mention may also be made of fibers coated with mineral or organic pigments, such as the pigments described herein below.

For example, in another embodiment of the present disclosure, fibers of synthetic origin, for instance organic fibers, such as those used in surgery, are used.

For further example, in still another embodiment of the present disclosure, the fibers may be chosen from polyamide fibers, cellulose fibers and polyethylene fibers. Their length (L) may range from 0.1 mm to 5 mm such as from 0.25 mm to 1.6 mm, and their mean diameter may range from 1 μm to 50 μm. For instance, the polyamide fibers sold by Etablissements P. Bonte under the name “Polyamide 0.9 Dtex 3 mm,” having a mean diameter of 6 μm, a yarn count of about 0.9 dtex and a length ranging from 0.3 mm to 5 mm, or the polyamide fibers sold under the name Fiberlon 931-D1-S by the company LCW, having a yarn count of about 0.9 dtex and a length of about 0.3 mm, may be used. Cellulose (or rayon) fibers with a mean diameter of 50 μm and a length ranging from 0.5 mm to 6 mm may also be used, for instance those sold under the name “Natural rayon flock fiber RC1BE-N003-M04” by the company Claremont Flock. Polyethylene fibers, for instance those sold under the name “Shurt Stuff 13 099 F” by the company Mini Fibers, may also be used.

Elastomeric fibers may also be used, i.e. fibers which, when subjected to a stretching stress (for example of 30% relative to their initial length), return to a length substantially identical to their initial length when the stress is removed. Among the elastomeric fibers that may be used, non-limiting mention may be made of polyurethane fibers such as elastane (or Spandex®), fibers comprising at least 85% by weight of segmented polyurethane, such as Lycra® sold by Dupont de Nemours, elastodiene, or alternatively rubbery fibers obtained from natural rubber. These elastomeric fibers may or may not be vulcanized.

The composition according to the present disclosure may also comprise “rigid” fibers, as opposed to the fibers mentioned above, which are not rigid fibers.

The rigid fibers, which are initially substantially straight, when placed in a dispersing medium, do not undergo a substantial change in shape, which is reflected by the angular condition defined below, reflecting a shape that may be described as still substantially straight and linear. This angle condition reflects the stiffness of the fibers, which it is difficult to express by another parameter for objects that are as small as the rigid fibers.

The stiffness of the fibers is reflected by the following angular condition: for example, at least 50%, for instance at least 75%, such as at least 90%, in numerical terms, of the rigid fibers are such that the angle formed between the tangent to the longitudinal central axis of the fiber and the straight line connecting the said end to the point on the longitudinal central axis of the fiber corresponding to half the length of the fiber is less than 15°, and the angle formed between the tangent to the longitudinal central axis of the fiber at a point half way along the fiber and the straight line connecting one of the ends to the point on the longitudinal central axis of the fiber corresponding to half the length of the fiber, is less than or equal to 15° for the same fiber length ranging from 0.8 mm to 5 mm, for instance ranging from 1 mm to 4 mm, and from 1 mm to 3 mm, such as 2 mm.

For example, the angle mentioned above may be measured at the two ends of the fiber and at a point half way along the fiber; in other words, three measurements can be taken in this case and the average of the measured angles is less than or equal to 15°.

The tangent, at any point on the fiber, for instance forms an angle of less than 15°.

In the present disclosure, the angle formed by the tangent at a point on the fiber is the angle formed between the tangent to the longitudinal central axis of the fiber at the said point on the fiber and the straight line connecting the end of the fiber that is closest to the said point to the point on the longitudinal central axis of the fiber corresponding to half the length of the fiber.

For example, the rigid fibers that may be used in the composition as disclosed herein can have the same or substantially the same fiber length.

For instance, when a medium where rigid fibers are dispersed in an amount of 1% by weight, is observed by microscope, with an objective lens allowing a magnification of 2.5 and with full-field vision, a numerical majority of the rigid fibers, i.e. at least 50% numerically of the rigid fibers, for instance, at least 75% numerically of the rigid fibers such as at least 90% numerically of the rigid fibers, must satisfy the angular condition defined above. The measurement leading to the angle value is performed for the same length of fibers, this length ranging from 0.8 mm to 5 mm, for instance from 1 to 4 mm, and from 1 to 3 mm, such as 2 mm.

The medium on which the observation is performed is a dispersing medium that ensures good dispersion of the rigid fibers, for example water or an aqueous gel of clay or of associative polyurethane. A direct observation of the composition comprising the rigid fibers may even be performed. A sample of the prepared composition or dispersion is placed between a slide and cover slip for observation by microscope with an objective lens allowing a magnification of 2.5 and with full-field vision. Full-field vision allows the fibers to be viewed in their entirety.

The rigid fibers can be chosen from fibers of a synthetic polymer chosen from polyesters, polyurethanes, acrylic polymers, polyolefins, polyamides, for instance nonaromatic polyamides, and aromatic polyimideamides.

Non-limiting examples of rigid fibers that may be mentioned include:
polyester fibers, such as those obtained by chopping yarns sold under the names Fiber 255-100-R11-242T Taille 3 mm (eight-lobed cross section), Fiber 265-34-R11-56T Taille 3 mm (round cross section) and Fiber Coolmax 50-34-591 Taille 3 mm (four-lobed cross section) by the company Dupont de Nemours;
polyamide fibers, such as those sold under the names Trilobal Nylon 0.120-1.8 DPF; Trilobal Nylon 0.120-18 DPF; Nylon 0.120-6 DPF by the company Cellusuede products; or obtained by chopping yarns sold under the name Fiber Nomex Brand 430 Taille 3 mm by the company Dupont de Nemours;
polyimideamide fibers, such as those sold under the names “Kermel” and “Kermel Tech” by the company RHODIA;
poly(p-phenyleneterephthalamide) (or aramide) sold, for instance, under the name Kevlar® by the company Dupont de Nemours;
fibers with a multilayer structure comprising alternating layers of polymers chosen from polyesters, acrylic polymers and polyamides, such as those described in European Patent Nos. EP-A-6 921 217 and EP-A-686 858, and U.S. Pat. No. 5,472,798. Such fibers are sold under the names “Morphotex” and “Teijin Tetron Morphotex” by the company Teijin.

In one embodiment of the present disclosure, the rigid fibers are chosen from aromatic polyimideamide fibers.

Polyimideamide, yarns or fibers that may be used for the compositions of the present disclosure are described, for example, in the article from R. Pigeon and P. Allard, Chimie Macromoléculaire Appliquée, 40/41 (1974), pages 139-158 (No. 600), or in U.S. Pat. No. 3,802,841, French Patent No. FR-A-2 079 785, and European Patent Nos. EP-A1-0 360 728 and EP-A-0 549 494, which are incorporated herein by reference.

In one aspect of the present disclosure, the aromatic polyimideamide fibers are polyimideamide fibers comprising repeating units of formula: 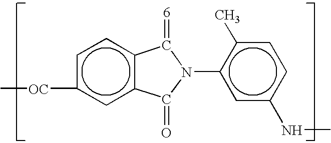
obtained by polycondensation of tolylene diisocyanate and trimellitic anhydride.

The fibers are present in the composition according to the present disclosure in an amount ranging from 0.05% to 10% by weight, for instance from 0.1% to 5% by weight, such as from 0.3% to 3% by weight, relative to the total weight of the composition.

The composition as disclosed herein may further comprise at least one additional wax, other than the at least one tacky microcrystalline wax.

The additional waxes that may be used in the compositions according to the present disclosure are chosen from waxes that are solid and rigid at room temperature, of animal, plant, mineral or synthetic origin, and mixtures thereof.

The at least one additional wax may also have a hardness ranging from 0.05 MPa to 30 MPa, for instance ranging from 6 MPa to 15 MPa, the hardness being determined by the method mentioned for the tacky wax.

Among the additional waxes that may be used, non-limiting mention may be made of hydrocarbon-based waxes such as beeswax, lanolin wax and Chinese insect waxes; rice wax, carnauba wax, candelilla wax, ouricury wax, esparto grass wax, cork fiber wax, sugar cane wax, Japan wax and sumach wax; montan wax, microcrystalline waxes, paraffins and ozokerite; polyethylene waxes, the waxes obtained by Fisher-Tropsch synthesis and waxy copolymers, and also esters thereof. Non-limiting mention may also be made of the waxes obtained by catalytic hydrogenation of animal or plant oils comprising linear or branched C8-C32 fatty chains.

Among these, further non-limiting mention may be made of, for example, hydrogenated jojoba oil, isomerized jojoba oil such as the partially hydrogenated isomerized jojoba oil manufactured or sold by the company Desert Whale under the commercial reference Iso-Jojoba-50, hydrogenated sunflower oil, hydrogenated castor oil, hydrogenated coconut oil and hydrogenated lanolin oil, bis(1,1,1-trimethylolpropane)tetrastearate sold under the name “Hest 2T-4S” by the company Heterene and bis(1,1,1-trimethylolpropane) tetrabehenate sold under the name Hest 2T-4B by the company Heterene.

Non-limiting mention may also be made of silicone waxes and fluoro waxes.

It is also possible to use the wax obtained by hydrogenation of olive oil esterified with stearyl alcohol, sold under the name “Phytowax Olive 18 L 57” or the waxes obtained by hydrogenation of castor oil esterified with cetyl alcohol, sold under the name “Phytowax ricin 16L64 and 22L73” by the company Sophim. Such waxes are described in French Patent Application No. FR-A-2 792 190.

The at least one additional wax may also be present in the form of a wax microdispersion as described above for the tacky wax. The at least one additional wax, when present, can be present in the composition according to the present disclosure in an amount ranging from 0.1% to 50%, for instance from 0.5% to 30%, such as from 1% to 20% by weight, relative to the total weight of the composition.

Cosmetically Acceptable Medium

The cosmetically acceptable medium of the composition may comprise at least one volatile solvent chosen, for example, from the volatile organic solvents and volatile oils defined below, and mixtures thereof.

The composition according to the present disclosure can comprise an aqueous medium, constituting an aqueous phase, which may form the continuous phase of the composition. The aqueous phase may consist of water, or it may comprise a mixture of water and of at least one water-miscible solvent (water miscibility of greater than 50% by weight at 25° C.), for instance lower monoalcohols comprising from 1 to 5 carbon atoms, such as ethanol or isopropanol, glycols comprising from 2 to 8 carbon atoms, such as propylene glycol, ethylene glycol, 1,3-butylene glycol or dipropylene glycol, C₃-C₄ ketones and C₂-C₄ aldehydes.

The aqueous phase (water and optionally the at least one water-miscible organic solvent) can be present in an amount ranging from 1% to 95% by weight, for instance, ranging from 3% to 80% by weight, such as ranging from 5% to 60% by weight, relative to the total weight of the composition.

The composition according to the present disclosure may comprise an oil or organic solvent that may form, for example, a fatty phase, such as a continuous fatty phase. The composition may be an anhydrous composition.

As used herein, the expression “volatile oil or organic solvent,” is understood to mean any non-aqueous medium that can evaporate on contact with the keratin fiber in less than one hour at room temperature and atmospheric pressure. The volatile organic solvents and the volatile oils that may be used as disclosed herein are volatile cosmetic organic solvents and oils, that are liquid at room temperature, having a non-zero vapor pressure at room temperature and atmospheric pressure, ranging from 0.13 Pa to 40 000 Pa (10⁻³ mmHg to 300 mmHg), for instance, ranging from 1.3 Pa to 13,000 Pa (0.01 mmHg to 100 mmHg) such as ranging from 1.3 Pa to 1 300 Pa (0.01 mmHg to 10 mmHg).

As used herein, the expression “non-volatile oil,” is understood to mean an oil that remains on the keratin fiber at room temperature and atmospheric pressure for at least several hours and which, for example, can have a vapor pressure of less than 10⁻³ mmHg (0.13 Pa). These oils may be hydrocarbon-based oils, silicone oils, or mixtures thereof.

As used herein, the expression “hydrocarbon-based oil,” is understood to mean an oil mainly comprising hydrogen and carbon atoms and optionally oxygen, nitrogen, sulphur or phosphorus atoms. The volatile hydrocarbon-based oils may be chosen from hydrocarbon-based oils comprising from 8 to 16 carbon atoms, for example, C₈-C₁₆ branched alkanes, for instance C₈-C₁₆ isoalkanes of petroleum origin (also known as isoparaffins), for instance isododecane (also known as 2,2,4,4,6-pentamethylheptane), isodecane and isohexadecane, and, for example, the oils sold under the trade names Isopars or Permetyls, C₈-C₁₆ branched esters, isohexyl neopentanoate, and mixtures thereof. Other volatile hydrocarbon-based oils may also be used, for instance petroleum distillates, such as those sold under the name Shell Solt by the company Shell, may also be used. The volatile solvent for example, may be chosen from hydrocarbon-based volatile oils comprising from 8 to 16 carbon atoms, and mixtures thereof.

Volatile oils which may also be used are volatile silicones such as, for example, linear or cyclic volatile silicone oils, for instance those with a viscosity≦6 centistokes (6×10⁻⁶ m²/s) and comprising, for example, from 2 to 10 silicon atoms, these silicones optionally comprising alkyl or alkoxy groups comprising from 1 to 22 carbon atoms. Among the volatile silicone oils that may be used in the present disclosure, non-limiting mention may be made, for example, of octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.

The at least one volatile oil, when present, can be present in the composition as disclosed herein in an amount ranging from 0.1% to 98% by weight, such as ranging from 1% to 65% by weight, relative to the total weight of the composition.

The composition can also comprise at least one non-volatile oil chosen, for instance, from non-volatile hydrocarbon-based oils and/or silicone oils.

Non-limiting examples of non-volatile hydrocarbon-based oils that may be used include:
hydrocarbon-based plant oils such as triglycerides comprising of fatty acid esters and glycerol in which the fatty acids may have varied chain lengths from C₄ to C₂₄, these chains possibly being linear or branched, and saturated or unsaturated; these oils are, for instance, wheat germ oil, sunflower oil, grape seed oil, sesame oil, corn oil, apricot oil, castor oil, shea butter, avocado oil, olive oil, soybean oil, sweet almond oil, palm oil, rapeseed oil, cotton oil, hazelnut oil, macadamia oil, jojoba oil, alfalfa oil, poppy oil, pumpkin oil, marrow oil, blackcurrant seed oil, evening primrose oil, millet oil, barley oil, quinoa oil, rye oil, safflower oil, candlenut oil, passion flower oil and musk rose oil; or alternatively caprylic/capric acid triglycerides such as those sold by Stéarineries Dubois or those sold under the names Miglyol 810, 812 and 818 by Dynamit Nobel;
synthetic ethers comprising from 10 to 40 carbon atoms;
linear or branched hydrocarbons of mineral or synthetic origin, such as petroleum jelly, polydecenes, hydrogenated polyisobutene such as parleam, and squalane, and mixtures thereof;
synthetic esters such as oils of formula R₁COOR₂ wherein R₁ is chosen from linear and branched fatty acid residues comprising from 1 to 40 carbon atoms and R₂ is chosen from, for example, branched hydrocarbon-based chains comprising from 1 to 40 carbon atoms, on the condition that R₅+R₆≧10, such as, for example, purcellin oil (cetostearyl octanoate), isopropyl myristate, isopropyl palmitate, C₁₂-C₁₅ alkyl benzoate, hexyl laurate, diisopropyl adipate, isononyl isononanoate, 2-ethylhexyl palmitate, isostearyl isostearate, alkyl or polyalkyl octanoates, decanoates or ricinoleates such as propylene glycol dioctanoate; hydroxylated esters such as isostearyl lactate and diisostearyl malate; and pentaerythritol esters;
fatty alcohols that are liquid at room temperature, comprising a branched and/or unsaturated carbon-based chain comprising from 12 to 26 carbon atoms, for instance octyidodecanol, isostearyl alcohol, oleyl alcohol, 2-hexyldecanol, 2-butyloctanol or 2-undecylpentadecanol;
higher fatty acids such as oleic acid, linoleic acid or linolenic acid; and mixtures thereof.

The non-volatile silicone oils which may be used in the composition according to the present disclosure may be non-volatile polydimethylsiloxanes (PDMSs), polydimethylsiloxanes comprising alkyl or alkoxy groups, that are pendent and/or at the end of a silicone chain, the groups each comprising from 2 to 24 carbon atoms, phenylsilicones, for instance phenyltrimeth icones, phenyldimethicones, phenyltrimethylsiloxydiphenylsiloxanes, diphenyldimethicones, diphenylmethyldiphenyltrisiloxanes and 2-phenylethyl trimethylsiloxysilicates.

The at least one non-volatile oil, when present, may be present in the composition as disclosed herein in an amount ranging from 0% to 30% (such as from 0.1% to 30%) by weight, for instance from 0% to 20% by weight (such as 0.1% to 20%) and for example, from 0 to 10% by weight (such as from 0.1% to 10%), relative to the total weight of the composition.

The composition according to the present disclosure may also comprise at least one fatty compound that is pasty at room temperature. As used herein, the expression “pasty fatty substance,” is understood to mean fatty substances with a melting point ranging from 20° C. to 55° C., for instance from 25° C. to 45° C., and/or a viscosity at 40° C. ranging from 0.1 Pa·s to 40 Pa·s (1 to 400 poises), such as from 0.5 Pa·s to 25 Pa·s, measured using a Contraves TV or Rheomat 80 viscometer, equipped with a spindle rotating at 60 Hz. A person skilled in the art can select the spindle for measuring the viscosity from the spindles MS-r3 and MS-r4, on the basis of general knowledge, so as to be able to carry out the measurement of the pasty compound tested.

These fatty substances may be, for example, hydrocarbon-based compounds, optionally of polymeric type; they can also be chosen from silicone compounds; they may also be in the form of a mixture of hydrocarbon-based compounds and/or silicone compounds. In the case of a mixture of different pasty fatty substances, the hydrocarbon-based pasty compounds (comprising mainly hydrogen and carbon atoms and optionally ester groups) may be, for instance, used in major proportion.

Among the pasty compounds which may be used in the composition according to the present disclosure, non-limiting mention may be made of lanolins and lanolin derivatives such as acetylated lanolins or oxypropylenated lanolins or isopropyl lanolate, having a viscosity ranging from 18 Pa·s to 21 Pa·s, such as from 19 Pa·s to 20.5 Pa·s, and/or a melting point ranging from 30° C. to 55° C., and mixtures thereof. It is also possible to use esters of fatty acids or of fatty alcohols, for example, those comprising from 20 to 65 carbon atoms (melting point ranging from 20° C. to 35° C. and/or viscosity at 40° C. ranging from 0.1 Pa·s to 40 Pa·s), such as triisostearyl or cetyl citrate; arachidyl propionate; polyvinyl laurate; cholesterol esters, such as triglycerides of plant origin, such as hydrogenated plant oils, viscous polyesters such as poly(12-hydroxystearic acid), and mixtures thereof.

Further non-limiting mention may also be made of pasty silicone fatty substances such as polydimethylsiloxanes (PDMSs) comprising pendent chains of the alkyl or alkoxy type comprising from 8 to 24 carbon atoms, and having a melting point of ranging from 20° C.-55° C., such as stearyldimethicones, for instance, those sold by Dow Corning under the trade names DC2503 and DC25514, and mixtures thereof.

The at least one pasty fatty substance, when present, can be present in the composition as disclosed herein in an amount ranging from 0.01% to 60% by weight, relative to the total weight of the composition, for instance, in an amount ranging from 0.5% to 45% by weight, such as ranging from 2% to 30% by weight, relative to the total weight of the composition.

The composition according to the present disclosure can further comprise at least one emulsifying surfactant, present for example, in an amount ranging from 2% to 30% by weight, relative to the total weight of the composition, for instance from 5% to 15%. These surfactants may be chosen from anionic and nonionic surfactants. Reference may be made to the document Encyclopedia of Chemical Technology, Kirk-Othmer, volume 22, pp. 333-432, 3rd edition, 1979, Wiley, for the definition of the properties and functions (emulsifying) of surfactants, for example, pp. 347-377 of the reference, for the anionic and nonionic surfactants.

Among the surfactants that can be used in the composition according to the present disclosure, non-limiting mention may be made of those chosen from:
nonionic surfactants: fatty acids, fatty alcohols, polyethoxylated or polyglycerolated fatty alcohols such as polyethoxylated stearyl or cetylstearyl alcohol, fatty acid esters of sucrose, alkylglucose esters, for instance polyoxyethylenated fatty esters of C₁-C₆ alkyl glucose, and mixtures thereof; and
anionic surfactants: C₁₆-C₃₀ fatty acids neutralized with amines, aqueous ammonia or alkaline salts, and mixtures thereof.

In one embodiment of the present disclosure, surfactants that make it possible to obtain an oil-in-water or wax-in-water emulsion are used.

The composition according to the present disclosure can also comprise at least one film-forming polymer.

The at least one film-forming polymer, when present, can be present in the composition as disclosed herein in a solids content amount ranging from 0.1% to 60% by weight, for instance from 0.5% to 40% by weight, such as from 1% to 30% by weight, relative to the total weight of the composition.

As used herein, the expression “film-forming polymer,” is understood to mean a polymer that is capable, by itself or in the presence of an auxiliary film-forming agent, of forming a continuous and adherent film on a support, for instance on keratin materials such as the eyelashes.

Among the film-forming polymers that may be used in the composition of the present disclosure, non-limiting mention may be made of synthetic polymers, of radical-mediated type or of polycondensate type, and polymers of natural origin, and mixtures thereof.

As used herein, the expression “radical-mediated film-forming polymer,” is understood to mean a polymer obtained by polymerization of monomers comprising unsaturation, for instance ethylenic unsaturation, each monomer being capable of homopolymerizing (unlike polycondensates). The film-forming polymers of radical-mediated type may be, for example, vinyl polymers or copolymers, such as acrylic polymers.

The vinyl film-forming polymers can result from the polymerization of monomers comprising ethylenic unsaturation and comprising at least one acidic group and/or esters of these acidic monomers and/or amides of these acidic monomers.

Monomers bearing an acidic group which may be used include α,β-ethylenic unsaturated carboxylic acids such as acrylic acid, methacrylic acid, crotonic acid, maleic acid or itaconic acid. In one embodiment of the present disclosure, (Meth)acrylic acid and crotonic acid are used, and in some instances, (meth)acrylic acid.

The esters of acidic monomers may be chosen from, for example, (meth)acrylic acid esters (also known as (meth)acrylates), for instance, (meth)acrylates of an alkyl, such as of a C₁-C₃₀ and for instance, C₁-C₂₀ alkyl, (meth)acrylates of an aryl, such as of a C₆-C₁₀ aryl, and (meth)acrylates of a hydroxyalkyl, for instance of a C₂-C₆ hydroxyalkyl. Among the alkyl (meth)acrylates that may be used, non-limiting mention may be made of methyl methacrylate, ethyl methacrylate, butyl methacrylate, isobutyl methacrylate, 2-ethylhexyl methacrylate, lauryl methacrylate and cyclohexyl methacrylate.

Among the hydroxyalkyl (meth)acrylates that may be used, non-limiting mention may be made of hydroxyethyl acrylate, 2-hydroxypropyl acrylate, hydroxyethyl methacrylate and 2-hydroxypropyl methacrylate. Among the aryl (meth)acrylates that may be used, non-limiting mention may be made of benzyl acrylate and phenyl acrylate.

In one embodiment of the present disclosure, the (meth)acrylic acid esters used are alkyl meth)acrylates.

According to the present disclosure, the alkyl group of the esters may be either fluorinated or perfluorinated, i.e. some or all of the hydrogen atoms of the alkyl group are substituted with fluorine atoms.

Non-limiting examples of amides of the acid monomers that may be mentioned include (meth)acrylamides, for instance N-alkyl(meth)acrylamides, such as of a C₂-C₁₂ alkyl. Among the N-alkyl(meth)acrylamides that may be used, non-limiting mention may be made of N-ethylacrylamide, N-t-butylacrylamide, N-t-octylacrylamide and N-undecylacrylamide.

The vinyl film-forming polymers may also result from the homopolymerization or copolymerization of monomers chosen from vinyl esters and styrene monomers. For example, these monomers may be polymerized with acid monomers and/or esters thereof and/or amides thereof, such as those mentioned above. Non-limiting examples of vinyl esters that may be mentioned include vinyl acetate, vinyl neodecanoate, vinyl pivalate, vinyl benzoate and vinyl t-butylbenzoate. Non-limiting examples of styrene monomers that may be mentioned are styrene and α-methylstyrene.

Among the film-forming polycondensates that may be used, non-limiting mention may be made of polyurethanes, polyesters, polyesteramides, polyamides, epoxyester resins and polyureas. The polyurethanes can be chosen from anionic, cationic, nonionic and amphoteric polyurethanes, polyurethane-acrylics, polyurethane-polyvinylpyrrolidones, polyester-polyurethanes, polyether-polyurethanes, polyureas and polyurea/polyurethanes, and mixtures thereof. The polyesters may be obtained, in a known manner, by polycondensation of dicarboxylic acids with polyols, such as diols. The dicarboxylic acid may be aliphatic, alicyclic or aromatic. Non-limiting examples of such acids that may be mentioned include: oxalic acid, malonic acid, dimethylmalonic acid, succinic acid, glutaric acid, adipic acid, pimelic acid, 2,2-dimethylglutaric acid, azelaic acid, suberic acid, sebacic acid, fumaric acid, maleic acid, itaconic acid, phthalic acid, dodecanedioic acid, 1,3-cyclohexanedicarboxylic acid, 1,4-cyclohexanedicarboxylic acid, isophthalic acid, terephthalic acid, 2,5-norbornanedicarboxylic acid, diglycolic acid, thiodipropionic acid, 2,5-naphthalenedicarboxylic acid and 2,6-naphthalenedicarboxylic acid. These dicarboxylic acid monomers may be used alone or as a combination of at least two dicarboxylic acid monomers. In one embodiment of the present disclosure, the monomers are chosen from phthalic acid, isophthalic acid and terephthalic acid. The diol may be chosen from aliphatic, alicyclic and aromatic diols. For example, the diol used may be chosen from: ethylene glycol, diethylene glycol, triethylene glycol, 1,3-propanediol, cyclohexanedimethanol and 4-butanediol. Other polyols that may be used are glycerol, pentaerythritol, sorbitol and trimethylolpropane.

The polyesteramides may be obtained in a manner analogous to that of the polyesters, by polycondensation of diacids with diamines or amino alcohols. Diamines that may be used include ethylenediamine, hexamethylenediamine and meta- or para-phenylenediamine. An amino alcohol that may be used is monoethanolamine.

The polyester may also comprise at least one monomer bearing at least one group —SO₃M, wherein M ris chosen from a hydrogen atom, an ammonium ion NH₄⁺ and metal ions, such as, for example, Na⁺, Li⁺, K⁺, Mg²⁺, Ca²⁺, Cu²⁺, Fe²⁺ or Fe³⁺ ions. In one embodiment of the present disclosure, a difunctional aromatic monomer comprising such a group —SO₃M is used. The aromatic nucleus of the difunctional aromatic monomer also bearing a group —SO₃M as described above may be chosen, for example, from benzene, naphthalene, anthracene, biphenyl, oxybiphenyl, sulphonylbiphenyl and methylenebiphenyl nuclei. As non-limiting examples of difunctional aromatic monomers also bearing a group —SO₃M, mention may be made of: sulphoisophthalic acid, sulphoterephthalic acid, sulphophthalic acid, and 4-sulphonaphthalene-2,7-dicarboxylic acid.

For example, the copolymers may be chosen from those based on isophthalate/sulphoisophthalate, for instance copolymers obtained by condensation of diethylene glycol, cyclohexanedimethanol, isophthalic acid and sulphoisophthalic acid.

The polymers of natural origin, optionally modified, may be chosen from shellac resin, sandarac gum, dammar resins, elemi gums, copal resins and cellulose polymers, and mixtures thereof.

According to one embodiment of the composition according to the present disclosure, the at least one film-forming polymer can be a water-soluble polymer and may be present in an aqueous phase of the composition; the polymer is thus solubilized in the aqueous phase of the composition. Non-limiting examples of water-soluble film-forming polymers that may be mentioned are:
proteins, for instance proteins of plant origin such as wheat proteins and soybean proteins; proteins of animal origin such as keratins, for example keratin hydrolyzates and sulphonic keratins;
polymers of cellulose such as hydroxyethylcellulose, hydroxypropylcellulose, methylcellulose, ethylhydroxyethylcellulose and carboxymethylcellulose, and quaternized cellulose derivatives;
acrylic polymers or copolymers, such as polyacrylates or polymethacrylates;
vinyl polymers, for instance polyvinylpyrrolidones, copolymers of methyl vinyl ether and of malic anhydride, the copolymer of vinyl acetate and of crotonic acid, copolymers of vinylpyrrolidone and of vinyl acetate; copolymers of vinylpyrrolidone and of caprolactam; polyvinyl alcohol;
polymers of natural origin, which are optionally modified, such as:
gum arabics, guar gum, xanthan derivatives, karaya gum;
alginates and carrageenans;
glycosaminoglycans, hyaluronic acid and derivatives thereof;
shellac resin, sandarac gum, dammar resins, elemi gums and copal resins;
deoxyribonucleic acid;
mucopolysaccharides such as chondroitin sulphate, and mixtures thereof.

According to another embodiment of the composition as disclosed herein, the at least one film-forming polymer may be a polymer dissolved in a liquid fatty phase comprising organic solvents or oils such as those described above (the film-forming polymer is thus said to be a liposoluble polymer). As used herein, the expression “liquid fatty phase,” is understood to mean a fatty phase which is liquid at room temperature (25° C.) and atmospheric pressure (760 mmHg, i.e. 10⁵ Pa), composed of at least one fatty substance that is liquid at room temperature, also known as oils, which are generally mutually compatible.

The liquid fatty phase may comprise, for example, a volatile oil, optionally mixed with a non-volatile oil, the oils possibly being chosen from those mentioned above.

Non-limiting examples of liposoluble polymers that may be mentioned include copolymers of vinyl ester (the vinyl group being directly linked to the oxygen atom of the ester group and the vinyl ester comprising a saturated, linear or branched hydrocarbon-based radical of 1 to 19 carbon atoms, linked to the carbonyl of the ester group) and of at least one other monomer chosen from vinyl esters (other than the vinyl ester already present), α-olefins (comprising from 8 to 28 carbon atoms), alkyl vinyl ethers (in which the alkyl group comprises from 2 to 18 carbon atoms) and allylic and methallylic esters (comprising a saturated, linear or branched hydrocarbon-based radical of 1 to 19 carbon atoms, linked to the carbonyl of the ester group).

These copolymers may be crosslinked with the aid of crosslinking agents, which may be either of the vinyl type or of the allylic or methallylic type, such as tetraallyloxyethane, divinylbenzene, divinyl octanedioate, divinyl dodecanedioate and divinyl octadecanedioate.

Non-limiting examples of these copolymers which may be mentioned include the following copolymers: vinyl acetate/allyl stearate, vinyl acetate/vinyl laurate, vinyl acetate/vinyl stearate, vinyl acetate/octadecene, vinyl acetate/octadecyl vinyl ether, vinyl propionate/allyl laurate, vinyl propionate/vinyl laurate, vinyl stearate/1-octadecene, vinyl acetate/1-dodecene, vinyl stearate/ethyl vinyl ether, vinyl propionate/cetyl vinyl ether, vinyl stearate/allyl acetate, vinyl 2,2-dimethyloctanoate/vinyl laurate, allyl 2,2-dimethylpentanoate/vinyl laurate, vinyl dimethylpropionate/vinyl stearate, allyl dimethylpropionate/vinyl stearate, vinyl propionate/vinyl stearate, crosslinked with 0.2% divinylbenzene, vinyl dimethylpropionate/vinyl laurate, crosslinked with 0.2% divinylbenzene, vinyl acetate/octadecyl vinyl ether, crosslinked with 0.2% tetaallyloxyethane, vinyl acetate/allyl stearate, crosslinked with 0.2% divinylbenzene, vinyl acetate/1-octadecene, crosslinked with 0.2% divinylbenzene, and allyl propionate/allyl stearate, crosslinked with 0.2% divinylbenzene.

Non-limiting examples of liposoluble film-forming polymers that may also be mentioned include liposoluble copolymers, for instance, those resulting from the copolymerization of vinyl esters comprising from 9 to 22 carbon atoms or of alkyl acrylates or methacrylates, and alkyl radicals comprising from 10 to 20 carbon atoms. Such liposoluble copolymers may be chosen from, for example, polyvinyl stearate, polyvinyl stearate crosslinked with the aid of divinylbenzene, of diallyl ether or of diallyl phthalate, polystearyl (meth)acrylate, polyvinyl laurate and polylauryl (meth)acrylate, it being possible for these poly(meth)acrylates to be crosslinked with the aid of ethylene glycol dimethacrylate or tetraethylene glycol dimethacrylate.

The liposoluble copolymers defined above are known and are described for instance, in French Patent Application FR-A-2 232 303; they can have a weight-average molecular weight ranging from 2,000 to 500,000, such as from 4,000 to 200,000.

Among the liposoluble film-forming polymers that may be used as disclosed herein, non-limiting mention may also be made of polyalkylenes, for example copolymers of C₂-C₂₀ alkenes, such as polybutene, alkylcelluloses with a linear or branched, saturated or unsaturated C₁-C₈ alkyl radical, for instance ethylcellulose and propylcellulose, copolymers of vinylpyrrolidone (VP), such as copolymers of vinylpyrrolidone and of C₂ to C₄₀ for instance C₃ to C₂₀ alkene. As non-limiting examples of VP copolymers that may be used in the present disclosure, non-limiting mention may be made of the copolymers of VP/vinyl acetate, VP/ethyl methacrylate, butylated polyvinylpyrrolidone (PVP), VP/ethyl methacrylate/methacrylic acid, VP/eicosene, VP/hexadecene, VP/triacontene, VP/styrene or VP/acrylic acid/lauryl methacrylate.

The at least one film-forming polymer may also be present in the composition in the form of particles dispersed in an aqueous phase or in a non-aqueous solvent phase, which is generally known as a latex or pseudolatex. The techniques for preparing these dispersions are well known to those skilled in the art.

Non-limiting examples of aqueous dispersions of film-forming polymers that may be used are the acrylic dispersions sold under the names Neocryl XK-90®, Neocryl A-1070®, Neocryl A-1090®, Neocryl BT-62®, Neocryl A-1079® and Neocryl A-523® by the company Avecia-Neoresins, Dow Latex 432® by the company Dow Chemical, Daitosol 5000 AD® by the company Daito Kasey Kogyo; or the aqueous dispersions of polyurethane sold under the names Neorez R-981® and Neorez R-974® by the company Avecia-Neoresins, Avalure UR-405®, Avalure UR-410®, Avalure UR-425®), Avalure UR-450®, Sancure 875®, Sancure 861®, Sancure 878® and Sancure 2060® by the company Goodrich, Impranil 85® by the company Bayer and Aquamere H-1511® by the company Hydromer; the sulphopolyesters sold under the brand name “Eastman AQ®”by the company Eastman Chemical Products, vinyl dispersions, for instance “Mexomer PAM” and also acrylic dispersions in isododecane, for instance “Mexomer PAP” by the company Chimex.

The composition according to the present disclosure may also comprise at least one plasticizer, which promotes the formation of a film with the film-forming polymer. Such a plasticizer may be chosen from any of the compounds known to those skilled in the art as being capable of satisfying the desired function.

Additives

The composition according to the present disclosure may also comprise at least one dyestuff, for instance pulverulent dyestuffs, liposoluble dyes and water-soluble dyes. The at least one dyestuff, when present, can be present in an amount ranging from 0.01% to 30% by weight, relative to the total weight of the composition.

The pulverulent dyestuffs may be chosen from pigments and nacres. The pigments may be white or coloured, mineral and/or organic, and coated or uncoated. Among the mineral pigments which may be used, non-limiting mention may be made of are titanium dioxide, optionally surface-treated, zirconium oxide, zinc oxide or cerium oxide, as well as iron oxide, chromium oxide, manganese violet, ultramarine blue, chromium hydrate and ferric blue. Among the organic pigments that may be used, non-limiting mention may be made of carbon black, pigments of D & C type, and lakes based on cochineal carmine or on barium, strontium, calcium or aluminium. The nacres may be chosen from white nacreous pigments such as mica coated with titanium or with bismuth oxychloride, colored nacreous pigments such as titanium mica with iron oxides, titanium mica with, for instance, ferric blue or chromium oxide, titanium mica with an organic pigment of the abovementioned type, and nacreous pigments based on bismuth oxychloride.

The liposoluble dyes may be chosen from, for example, Sudan Red, D&C Red 17, D&C Green 6, βcarotene, soybean oil, Sudan Brown, D&C Yellow 11, D&C Violet 2, D&C Orange 5, quinoline yellow and annatto. The water-soluble dyes may be chosen from, for example, beetroot juice, methylene blue, the disodium salt of ponceau, the disodium salt of alizarin green, quinoline yellow, the trisodium salt of amaranthus, the disodium salt of tartrazine the monosodium salt of rhodamine, the disodium salt of fuchsin, and xanthophyll.

The composition of the present disclosure may also comprise at least one additive chosen from any additives usually used in cosmetics, such as antioxidants, fillers, preserving agents, fragrances, neutralizers, thickeners and vitamins, and mixtures thereof.

The fillers may be chosen from those that are well known to a person skilled in the art and commonly used in cosmetic compositions. The fillers may be mineral or organic and lamellar or spherical. Non-limiting mention may be made of talc, mica, silica, kaolin, polyamide powder for instance Nylon® (Orgasol® from Atochem), poly-β-alanine powder and polyethylene powder, tetrafluoroethylene polymer powders for instance Teflon®, lauroyllysine, starch, boron nitride, expanded hollow polymer microspheres such as those made of polyvinylidene chloride/acrylonitrile, for instance Expancel® (Nobel Industrie), acrylic powders such as Polytrap® (Dow Corning), polymethyl methacrylate particles and silicone resin microbeads (for example Tospearls® from Toshiba), precipitated calcium carbonate, magnesium carbonate, magnesium hydrocarbonate, hydroxyapatite, hollow silica microspheres (Silica Beads® from Maprecos), glass or ceramic microcapsules, and metal soaps derived from organic carboxylic acids comprising from 8 to 22 carbon atoms, for instance, from 12 to 18 carbon atoms, for example zinc, magnesium or lithium stearate, zinc laurate or magnesium myristate.

The at least one filler, when present, can be present in an amount ranging from 0.1% to 25% such as from 1% to 20% by weight, relative to the total weight of the composition.

In one embodiment of the present disclosure, the composition is a mascara.

Needless to say, a person skilled in the art will take care to select the optional additional additives and/or the amount thereof such that the beneficial properties of the composition according to the present disclosure are not, or are not substantially, adversely affected by the addition envisaged.

The composition according to the present disclosure may be manufactured by the known processes generally used in cosmetics.

The composition according to the present disclosure may be packaged in an applicator product comprising a reservoir and a removable cap for closing the reservoir, for example in a leaktight manner.

The applicator assembly may also comprise a member for applying the makeup composition to the keratin fibers, such as the eyelashes, wherein the applicator member allows the composition to be taken up and also allows the composition taken up to be deposited on the eyelashes. This applicator member can be, for example, securely fastened to the cap for leaktight closure of the assembly.

The applicator assembly may also comprise a draining member (or drainer) for the applicator member, the draining member possibly being securely fastened to the reservoir.

The applicator member may for example, be a mascara brush that is well known to those skilled in the art. Such a brush for instance, comprises bristles arranged radially around a twisted core, such as a metal core. The brush may be of varied shape and may comprise cutout sections. Mascara brushes are described, for example, French Patent No. FR-A-2 607 373, and European Patent Nos. EP-A-611 170, EP-A-811 336, EP-A-811 337 and EP-A-842 620.

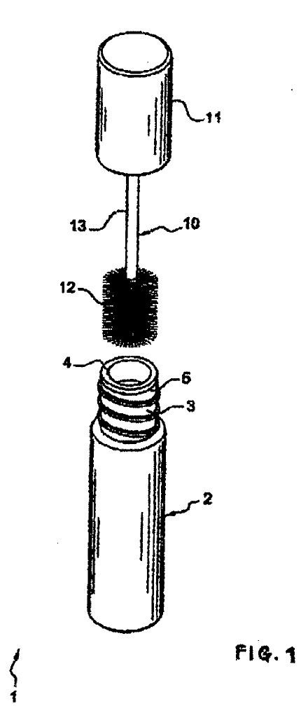

FIG. 1, to which reference is now made, shows one preferred embodiment of a packaging and applicator assembly 1 comprising a composition for coating keratin fibers according to the present disclosure. The packaging and applicator assembly 1 comprises a container 2 on which is mounted a threaded neck 3, one free edge of which delimits an aperture 4. In the aperture 4 is mounted a draining member 5. The assembly 1 also comprises an applicator device 10 comprising a cap 11 securely fastened to a stem 13, one end of which comprises an applicator 12, generally configured in the form of an arrangement of fibers held between the two branches of a twisted iron wire. An inner surface of the cap 11 is threaded so as to engage with the threading of the neck 3. Thus, when the applicator 12 and the stem 13 are placed inside the container 2, the threading of the cap 11 engages with the threading of the neck 3 such that the cap closes the aperture 4 of the container in a leaktight manner.

Alternatively, the applicator may comprise a comb comprising a plurality of teeth obtained by molding with a support made of thermoplastic material. The applicator may also comprise a comb combined with a brush.

Other than in the operating example, or where otherwise indicated, all numbers expressing quantities of ingredients, reaction conditions, and so forth used in the specification and claims are to be understood as being modified in all instances by the term “about.”. Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific example are reported as precisely as possible. Any numerical value, however, inherently contain certain errors necessarily resulting from the standard deviation found in their respective testing measurements. The example that follows serves to illustrate the invention without, however, being limiting in nature.

### EXAMPLE

A wax-in-water emulsion mascara having the composition below was prepared:

| | | |
|---|---|---|
| Microcrystalline wax SP18 from the company Strahl | 8 | g |
| and Pitsch | | |
| Beeswax | 1.85 | g |
| Carnauba wax | 1.2 | g |
| Wax obtained by hydrogenation of olive oil esterified with | 2 | g |
| stearyl alcohol (Phytowax Olive 18 L 57) | | |
| Rice bran wax | 1.6 | g |
| Triethanolamine | 1.5 | g |
| Stearic acid | 3 | g |
| Glyceryl stearate | 3 | g |
| Hydroxyethylcellulose | 2 | g |
| PVP/Eicosene copolymer | 1.5 | g |
| Polyvinylpyrrolidone | 1 | g |
| Sodium Polymethacrylate (“Darvan 7” from Vanderbilt) | 1 | g |
| Mixture of aliphatic polyurethane, triethylamine and | 13.1 | g |
| water (38/1/61) (Avalure UR 450 from Noveon) | | |
| Modified maize starch as a 44% colloidal suspension in water | 0.95 | g |
| (KM13 Resin from Kama) | | |
| Quaternized hydroxyethylcellulose | 0.1 | g |
| (“Celquat SC 240C” from National Starch) | | |
| α-Gluconamidopropyldimethylhydroxyethylammonium | 0.1 | g |
| chloride as an aqueous 60% solution (Ceraphyl 60 | | |
| from ISP) | | |
| Oxyethylenated (20 EO)-oxypropylenated (20 PO) | 0.2 | g |
| polydimethylsiloxane mixture (Q2-5220 from Dow Corning) | | |
| Mixture of polydimethylsiloxane containing α-ω hydroxyl | 1 | g |
| groups and cyclopentadimethylsiloxane | | |
| Sorbitan sesquioleate | 0.25 | g |
| Hydrogenated polyisobutylene (Parleam SV from Nippon | 0.75 | g |
| Oil Fats) | | |
| Butylene glycol | 2 | g |
| Mixture of polydimethylsiloxane and hydrated silica (Mirasil | 0.2 | g |
| SM from Rhodia) | | |
| Cellulose fibers 1.3 mm long (Rayon Flock Rcise N0003 | 0.75 | g |
| MO4 from Claremont Flock Corporation) | | |
| Pigments | 6 | g |
| Ethanol | 1 | g |
| Preserving agents qs | | |
| Water qs | 100 | g |

This mascara was judged as forming a smooth and uniform makeup on eyelashes, and also as giving a volumizing and lengthening effect.

1. A composition for coating keratin fibers, comprising, in a cosmetically acceptable medium, fibers and at least one microcrystalline wax with a tack of greater than or equal to 0.7 N.s.

2. The composition according to claim 1, wherein the at least one microcrystalline wax has a tack ranging from 0.7 N.s to 30 N.s.

3. The composition according to claim 2, wherein the at least one microcrystalline wax has a tack ranging from 0.8 N.s to 5 N.s.

4. The composition according to claim 1, wherein the at least one microcrystalline wax has a hardness of less than or equal to 3.5 MPa.

5. The composition according to claim 4, wherein the at least one microcrystalline wax has a hardness ranging from 0.01 MPa to 3.5 MPa.

6. The composition according to claim 5, wherein the at least one microcrystalline wax has a hardness ranging from 0.2 MPa to 1 MPa.

7. The composition according to claim 1, wherein the at least one microcrystalline wax is present in an amount ranging from 0.5% to 60% by weight, relative to the total weight of the composition.

8. The composition according to claim 7, wherein the at least one microcrystalline wax is present in an amount ranging from 5% to 20% by weight, relative to the total weight of the composition.

9. The composition according to claim 1, wherein the fibers are chosen from silk fibers, cotton fibers; wool fibers, flax fibers, cellulose fibers, polyamide fibers, viscose fibers, acetate fibers, acrylic fibers, polyolefin fibers, silica fibers, carbon fibers, polytetrafluoroethylene fibers, insoluble collagen fibers, polyester fibers, polyvinyl chloride fibers, polyvinylidene chloride fibers, polyvinyl alcohol fibers, polyacrylonitrile fibers, chitosan fibers, polyurethane fibers, polyethylene phthalate fibers, fibers formed from mixtures of polymers, substantially rectilinear rigid fibers, elastomeric fibers, and mixtures thereof.

10. The composition according to claim 9, wherein the fibers are chosen from fibers of synthetic origin.

11. The composition according to claim 10, wherein the fibers are chosen from rigid fibers.

12. The composition according to claim 11, wherein the rigid fibers are chosen from aromatic polyimide-amide fibers.

13. The composition according to claim 12, wherein the polyimide-amide is obtained by polymerization of tolylene diisocyanate and trimellitic anhydride, and comprises repeating units of formula: 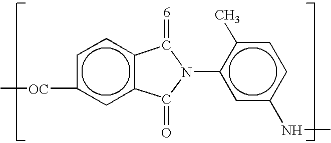
obtained by polycondensation of tolylene diisocyanate and trimellitic anhydride.

14. The composition according to claim 1, wherein the fibers have a length ranging from 1 μm to 10 mm.

15. The composition according to claim 14, wherein the fibers have a length ranging from 0.3 mm to 3 mm.

16. The composition according to claim 1, wherein the fibers have a cross section that is within a circle of diameter ranging from 2 nm to 500 μm.

17. The composition according to claim 16, wherein the fibers have a cross section that is within a circle of diameter ranging from 100 nm to 100 μm.

18. The composition according to claim 1, wherein the fibers are present in an amount ranging from 0.05% to 10% by weight, relative to the total weight of the composition.

19. The composition according to claim 18, wherein the fibers are present in an amount ranging from 0.3% to 3% by weight, relative to the total weight of the composition.

20. The composition according to claim 1, further comprising an aqueous phase.

21. The composition according to claim 20, wherein the aqueous phase comprises water or a mixture of water and of at least one water-miscible organic solvent.

22. The composition according to claim 21, wherein the at least one water-miscible organic solvent is chosen from lower monoalcohols comprising from 1 to 5 carbon atoms, glycols comprising from 2 to 8 carbon atoms, C₃-C₄ ketones and C₂-C₄ aldehydes.

23. The composition according to claim 20, wherein the aqueous phase is present in an amount ranging from 1% to 95% by weight, relative to the total weight of the composition.

24. The composition according to claim 23, wherein the aqueous phase is present in an amount ranging from 5% to 60% by weight, relative to the total weight of the composition.

25. The composition according to claim 1, further comprising at least one volatile oil.

26. The composition according to claim 25, wherein the at least one volatile oil is chosen from hydrocarbon-based oils and silicone oils.

27. The composition according to claim 25, wherein the at least one volatile oil is present in an amount ranging from 0.1% to 98% by weight, relative to the total weight of the composition.

28. The composition according to claim 27, wherein the at least one volatile oil is present in an amount ranging from 1% to 65% by weight, relative to the total weight of the composition.

29. The composition according to claim 1, further comprising at least one non-volatile oil.

30. The composition according to claim 29, wherein the at least one non-volatile oil is present in an amount ranging from 0.1% to 30% by weight, relative to the total weight of the composition.

31. The composition according to claim 30, wherein the at least one non-volatile oil is present in an amount ranging from 0.1% to 10% by weight, relative to the total weight of the composition.

32. The composition according to claim 1, further comprising at least one film-forming polymer.

33. The composition according to claim 32, wherein the at least one film-forming polymer is present in a solids content amount ranging from 0.1% to 60% by weight, relative to the total weight of the composition.

34. The composition according to claim 33, wherein the at least one film-forming polymer is present in a solids content amount ranging from 1% to 30% by weight, relative to the total weight of the composition.

35. The composition according to claim 1, further comprising at least one additional wax other than a microcrystalline wax.

36. The composition according to claim 35, wherein the at least one additional wax is present in the composition in an amount ranging from 0.1% to 50% by weight, relative to the total weight of the composition.

37. The composition according to claim 36, wherein the at least one additional wax is present in the composition in an amount ranging from 1% to 20% by weight, relative to the total weight of the composition.

38. The composition according to claim 1, further comprising at least one surfactant.

39. The composition according to claim 1, further comprising at least one additive chosen from dyestuffs, antioxidants, fillers, pasty fatty substances, preserving agents, fragrances, neutralizers, thickeners, vitamins, coalescers, plasticizers, and mixtures thereof.

40. The composition according to claim 1, wherein it is in the form of a mascara.

41. A non-therapeutic cosmetic process for making up and/or caring for keratin fibers, comprising applying to the keratin fibers a composition comprising, in a cosmetically acceptable medium, fibers and at least one microcrystalline wax with a tack of greater than or equal to 0.7 N.s.

42. The process according to claim 41, comprising applying the composition in an effective amount such that the keratin fibers, after the composition is applied, appear more uniform and/or smoother, and/or longer and/or thicker than the keratin fibers before the composition is applied.

43. A process for making up keratin fibers comprising applying to the keratin fibers a mascara composition comprising fibers and at least one microcrystalline wax with a tack of greater than or equal to 0.7 N.s and a hardness of less than or equal to 3.5 MPa,
wherein the mascara composition is applied in an amount effective to obtain a uniform and/or smooth makeup result on the keratin fibers, and a lengthening and/or volumizing effect on made-up keratin fibers.

44. An assembly for packaging and applying a product for coating keratin fibers, comprising:
i) a container comprising a composition comprising, in a cosmetically acceptable medium, fibers and at least one microcrystalline wax with a tack of greater than or equal to 0.7 N.s, and
ii) an applicator for applying the composition to the fibers.
